(19) United States
(12) Patent Application Publication
(10) Pub. No.: US 2014/0107218 A1
(43) Pub. Date: Apr. 17, 2014

(54) METHODS FOR MAINTAINING EYE HEALTH AND AMELIORATING OPTHALMIC MALADIES IN ANIMALS

(71) Applicant: NESTEC SA, Vevey (CH)

(72) Inventors: Scott Sherrill, Chesterfield, MO (US); Wei Wang, Creve Coeur, MO (US)

(73) Assignee: NESTEC SA, Vevey (CH)

(21) Appl. No.: 14/052,997

(22) Filed: Oct. 14, 2013

Related U.S. Application Data
(60) Provisional application No. 61/713,811, filed on Oct. 15, 2012.

(51) Int. Cl. A61K 31/132; A61K 45/06

(52) U.S. Cl. CPC A61K 31/132; A61K 45/06
USPC 514/674; 514/673

(57) ABSTRACT

The invention provides novel methods for maintaining eye health and ameliorating ophthalmic maladies in animals by administering to the animals an ophthalmic malady ameliorating amount of one or more polyamines. Generally, the polyamines are administered to the animals in amounts of from about 1 to about 200 mg/kg/day to ameliorate maladies such as cataracts and cloudy eye.

### CROSS REFERENCE TO RELATED APPLICATIONS

This application claims priority to U.S. Provisional Application No. 61/713,811 filed 15 Oct. 2012, the disclosure of which is incorporated herein by this reference.

### BACKGROUND OF THE INVENTION

1. Field of the Invention

The invention relates generally to methods for maintaining eye health and ameliorating ophthalmic maladies and particularly to methods for using polyamines to maintain eye health and ameliorate ophthalmic maladies in animals.

2. Description of Related Art

Polyamines are organic compounds having two or more primary amino groups. Polyamines are known to be synthesized in cells via highly-regulated pathways, but their actual cellular function is not entirely clear. Polyamines are believed to have anti-inflammatory and anti-oxidant properties, possibly by mediating the synthesis of anti-inflammatory proteins such as vasoregulin or by direct action on leucocytes and similar cells. Polyamines bind to negatively charged nucleic acids in cell membranes and stabilize membrane structures. Polyamines also act as scavengers of reactive oxygen species and various cellular structures that have incurred oxidative damage.

Ophthalmic maladies such as cataracts and cloudy eye are common in animals such as canines and felines, particularly animals that are outdoors and exposed to excessive sunlight and to aging animals that often develop cataracts, cloudy eye, and related conditions. Methods for counteracting some such ophthalmic maladies are known in the art. WO2011059474A1 discloses novel methods for maintaining eye health and ameliorating ophthalmic maladies in canines by administering to the canines an ophthalmic malady ameliorating amount of one or more carotenes. WO2009051223 discloses using vascular-adhesion-protein-1 (VAP-1) inhibitors for treating cataracts. US20090082415 discloses using a mixture of topical carriers having carbomer, glycerin, ethylene diamine tetraacetic acid, benzalkonium chloride, balance water, and aldose reductase inhibitors for treating optical complications of diabetes in dogs. U.S. Pat. No. 7,442,711 discloses using N-hydroxy piperidine compounds for the amelioration of cataracts and other ophthalmic diseases. US20060084685 discloses using roflumilast for the treatment of various eye diseases. These methods are, however, insufficient to solve all the problems associated with these and other ophthalmic maladies. There is, therefore, a need for new methods for ameliorating ophthalmic maladies in animals.

### SUMMARY OF THE INVENTION

It is, therefore, an object of the invention to provide methods for ameliorating ophthalmic maladies in animals.

It is another object of the invention to provide methods for maintaining eye health in animals.

It is a further object of the invention to provide methods for preventing or ameliorating cataracts in animals.

It is another object of the invention to provide methods for preventing or ameliorating cloudy eye in animals.

It is another object of the invention to provide methods for maintaining or improving vision in animals.

It is another object of the invention to provide methods for preventing or ameliorating animal eye injury in animals caused by radiation.

One or more of these other objects are achieved using novel methods for ameliorating ophthalmic maladies in animals. The methods comprise administering to the animals an ophthalmic malady ameliorating amount of one or more polyamines or one or more precursors that are synthesized in vivo to produce polyamines.

The polyamines are administered to the animals in amounts of from about [[[1 to about 2500 milligrams (mg) as desired or in amounts of from about 0.1 to about 500 mg per day.

Other and further objects, features, and advantages of the present invention will be readily apparent to those skilled in the art.

### DETAILED DESCRIPTION OF THE INVENTION

### Definitions

The term “polyamine” means any polyamine, polyamine functional analog, or any polyamine or polyamine functional analog synthesized in vivo by an animal from a polyamine precursor. For example, polyamines are derived in vivo from ornithine, e.g., by the enzyme ornithine decarboxylase. Similarly, polyamines are derived from arginine or citrulline after such amino acids are converted in vivo to ornithine and ornithine is converted in vivo to a polyamine. Therefore, “administering a polyamine” encompasses administering a polyamine or administering a polyamine precursor such as arginine, citrulline, or ornithine such that a polyamine will be synthesized in vivo.

The term “animal” means a human or other animal that could benefit from maintaining eye health or that is susceptible to or suffering from ophthalmic maladies, including cataracts and cloudy eye. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

The term “ophthalmic agent” means any compound, composition, or drug, other than polyamines, useful for ameliorating ophthalmic maladies in animals.

The term “in conjunction” means that one or more polyamines and one or more ophthalmic agents are administered to animals (1) together, e.g., in a food or pharmaceutical composition, or (2) separately at the same or different frequency using the same or different administration routes at about the same time or periodically. “Periodically” means that the agent is administered on a dosage schedule acceptable for a specific agent and that the polyamine is administered to animals routinely as appropriate for the particular animal. “About the same time” generally means that the polyamine and agent are administered at the same time or within about 72 hours of each other. “In conjunction” specifically includes administration schemes wherein an ophthalmic agent is administered for a prescribed period and polyamines are administered indefinitely.

The term “single package” means that the components of a kit are physically associated, in or with one or more containers, and considered a unit for manufacture, distribution, sale, or use. Containers include, but are not limited to, bags, boxes or cartons, bottles, packages of any type or design or material, over-wrap, shrink-wrap, affixed components (e.g., stapled, adhered, or the like), or combinations of any of the foregoing. For example, a single package kit may provide containers of individual compounds and/or compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain, for example, instructions on how to use the kit, or safety or technical information about one or more components of a kit. Examples of information that can be provided as part of a virtual kit include instructions for use; safety information such as material safety data sheets; poison control information; information on potential adverse reactions; clinical study results; dietary information such as food composition or caloric composition; general information on ophthalmic maladies; diseases that affect ophthalmic function, or general information on treatment or preservation of ophthalmic maladies; self-help relating to ophthalmic maladies; caregiver information for those caring for animals with ophthalmic maladies; and use, benefits, and potential side-effects or counter-indications for ophthalmic agents.

The term “health and wellness of an animal” means the complete physical, mental, and social well being of the animal, not merely the absence of disease or infirmity.

The term “quality of life” means the ability to enjoy normal life activities.

The term “extending the prime” means extending the number of years an animal lives a healthy life and not just extending the number of years an animal lives, e.g., an animal would be healthy in the prime of its life for a relatively longer time.

As used throughout, ranges are used herein in shorthand, so as to avoid having to set out at length and describe each and every value within the range. Any appropriate value within the range can be selected, where appropriate, as the upper value, lower value, or the terminus of the range.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “an animal”, “a method”, or “an agent” includes a plurality of such “animals”, “methods”, or “agents”. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Full citations for publications not cited fully within the specification are set forth at the end of the specification.

### The Invention

In one aspect, the invention provides methods for ameliorating ophthalmic maladies in animals. The methods comprise administering to the animals an ophthalmic malady ameliorating amount of one or more polyamines. The invention is based upon the unexpected discovery that the amount of polyamines in older animals is less than the amount of polyamines in younger animals and that polyamines administered to such older animals are effective for ameliorating changes to the eye associated with aging and damage to the eye caused by free radicals. While not bound by theory, it is believed that the polyamines reduce calcium ion induced protein aggregation, a possible mechanism for the polyamines protective effects for ameliorating ophthalmic maladies.

Generally, the ophthalmic maladies ameliorated by the invention are any maladies caused by aging or free radical damage to the eye. In one embodiment, the maladies are maladies caused by aging, e.g., cataracts or cloudy eye. In another embodiment, the maladies are maladies caused by radiation. Maladies known to be caused by radiation include cataracts, radiation retinopathy, photokeratitis, photoconjunctivitis, pterygium, pinguecula, and cancer. In a further embodiment, the maladies are maladies caused by free radicals. Maladies known to be caused by free radicals include cloudy eye, and cataracts.

The polyamines useful in the invention are any polyamines suitable for administration to animals. Such polyamines and their sources are known to skilled artisans. Generally, polyamines are obtained from any suitable source, synthetic, natural, and combinations thereof. A preferred source of polyamines is dietary polyamines contained in many polyamine-rich foods such as beef, pork, chicken, soybeans, mushrooms, orange leaves, green tea leaves, wheat germ, rice bran, beef intestines, liver of eel, black rice, turban shell viscera, mango, Japanese pumpkin, whelk, salted cod roe, nuts, fermented pickles, and the like. Other sources include synthetic polyamines, e.g., as dietary supplements in tablet, capsule, and similar forms.

In various embodiments, the polyamines are cadaverine, spermidine, spermine, putrescine, and combinations thereof. Preferably the polyamines are spermidine, spermine, putrescine, and combinations thereof. Most preferably, the polyamines are putrescine.

In certain embodiments, the polyamines are administered to the animals by administering a polyamine precursor to the animals and allowing the polyamines to be synthesized in vivo. Any polyamine precursor is suitable. In preferred embodiments, the polyamine precursor is arginine, lysine, citrulline, ornithine, or combinations thereof Putrescine is synthesized in vivo from the precursor arginine using two different pathways. In one, arginine is converted into agmatine with a reaction catalyzed by the enzyme arginine decarboxylase. Then, agmatine is transformed into carbamilputrescine by agmatine imino hydroxylase. Then, carbamilputrescine is converted into putrescine. In another, arginine is converted into ornithine and then ornithine is converted into putrescine by ornithine decarboxylase. Cadaverine is synthesized from lysine in a one-step reaction with lysine decarboxylase. Spermidine is synthesized from putrescine, using an aminopropylic group from decarboxylated S-adenosyl-L-methionine (SAM). The reaction is catalyzed by spermidine synthase. Spermine is synthesized from the reaction of spermidine with SAM in the presence of the enzyme spermidine synthase.

The polyamines are administered to the animals in any non-toxic amount that is effective for ameliorating ophthalmic maladies. In various embodiments, the polyamines are administered to the animal in amounts of from about 1 to about 200 milligrams per kilogram of body weight per day (mg/kg/day), preferably from about 10 to about 180 mg/kg/day), most preferably from about 20 to about 150 mg/kg/day. The amount may vary within the range depending on the malady, the polyamine administered, and the characteristics of the animal, e.g., size and age.

The polyamines are administered to the animals using any suitable method, e.g., parenterally, enterally (e.g., orally, nasally, and rectally), topically, or buccally. Preferably, the polyamines are administered orally, e.g., in a food composition. In various embodiments, administration is on an as-needed or as-desired basis of varying or regular frequency depending on the intended use. In other embodiments, administration is on a regular or scheduled basis. Administration on a regular or scheduled basis provides the animals with a regular and consistent dose of polyamines that keeps the level of polyamines in the eye relatively constant. Regular or scheduled administration can be once monthly, once weekly, once daily, or more than once daily. Similarly, administration can be every other day, week, or month, every third day, week, or month, every fourth day, week, or month, and the like. Administration can be multiple times per day. Such administration can depend on the age, size, health, malady, and the like for the animal. In some embodiments, polyamines or comestible ingredients containing polyamines are applied to or admixed with typical foods and drinks and administered to the animals, e.g., polyamines are an integral part of an animal pet food. In one embodiment, administration is part of a dietary regimen. For example, such a dietary regimen comprises the regular ingestion of one or more polyamines or any comestible ingredient containing such polyamines in an amount effective for ameliorating ophthalmic maladies. In preferred embodiments, the polyamines are administered to the animals as part of a food composition or in the form of a dietary supplement. Preferably, the polyamines are administered to the animals as part of a wet or dry pet food composition, e.g., kibbles or wet canned pet food. In a preferred embodiment, the polyamines are administered on an extended regular basis, preferably a daily basis.

In various embodiments, the animals are canines and felines, preferably canines. In a preferred embodiment, the animals are wolves, foxes, jackals, coyotes, and dogs, preferably dogs, most preferably domestic dogs.

Polyamine administration, including administration as part of a dietary regimen, can span a period ranging from fetus through the life of the animal. In certain embodiments, the animal is a young, growing, or adult animal. In preferred embodiments, the animal is an aging animal susceptible to or suffering from ophthalmic maladies characteristic of aging. In other embodiments, the animal is an animal that is likely to be exposed to excess radiation that will adversely impact the eye, e.g., a hunting dog or a working dog.

In one embodiment, the methods further comprise administering the polyamines in conjunction with one or more ophthalmic agents in an amount effective for ameliorating ophthalmic maladies as defined herein. In a particular embodiment, the polyamines are administered in a pharmaceutical composition that includes one or more ophthalmic agents along with one or more polyamines. In a preferred embodiment, the polyamines are administered to the animal on a daily basis, preferably as part of a food composition or in a dietary supplement, and the ophthalmic agents are administered on an as prescribed basis, generally in the form of a pharmaceutical composition such as a pill. Such agents include avastin (e.g., for radiation retinopathy); antibiotics; anti-inflammatory compounds (e.g., COX-2 inhibitors); tranquilizers; antioxidants such as resveratrol, proanthocyanidins, anthocyanins, carotenes, carotenoids (e.g., lutein, zeaxanthin, astaxanthin, α-carotene, β-carotene, cryptoxanthin, and lycopene); bioflavonoids (e.g., taxifolin, naringenin, and hesperetin); glutathione; catechin; epicatechin; epigallocatechin; epigallocatechin gallate; epicatechin gallate; and lipoic acid; and vitamins, e.g., vitamin A, vitamin E, and vitamin C. Skilled artisans can determine the proper type and amount of ophthalmic agents to administer to the animals based upon their age, size, health, malady, and the like for the animal.

Methods that ameliorate ophthalmic maladies have many beneficial effects for animals. Many day-to-day life functions are dependent upon or related to ophthalmic function. Eliminating or lessening the impact and severity of ophthalmic maladies that interfere with normal ophthalmic function, particularly those that impair vision, improve many aspects of life. For example, ameliorating ophthalmic maladies is related to (1) maintaining eye health; (2) maintaining or improving vision; (3) preventing or ameliorating cataracts; (4) preventing or ameliorating cloudy eye; (5) preventing or ameliorating animal eye injury caused by radiation; (6) reducing or preventing a decline of social interaction; (7) promoting overall health and wellness; (8) improving the quality of life; and (9) extending the prime. Therefore, in various aspects, the invention provides methods for affecting various ophthalmic related functions adversely affected by these ophthalmic maladies. In all these aspects, the benefit results from an accumulation of polyamines in the eyes of the animal, i.e., in the eye tissue exposed to radiation and susceptible to free radical damage.

In one such aspect, the invention provides methods for maintaining eye health in animals comprising administering to the animals an eye health maintaining amount of one or more polyamines. The polyamines maintain overall eye health by preventing ophthalmic maladies in animals, particularly those caused by radiation and by free radicals and elevated intra-lens calcium.

In another such aspect, the invention provides methods for maintaining or improving vision in animals comprising administering to the animals a vision maintaining or improving amount of one or more polyamines. The polyamines absorb radiation and scavenge free radicals and inhibit transglutaminase-catalyzed crystallin cross-linking that are at least partially responsible for causing ophthalmic maladies that impair vision. With no or less severe ophthalmic maladies, the animals have better vision.

In another aspect, the invention provides methods for preventing or ameliorating cataracts in animals comprising administering to the animals a cataract preventing or ameliorating amount of one or more polyamines. The polyamines absorb radiation and scavenge free radicals that are at least partially responsible for causing cataracts in animals. With no or less severe cataracts, the animals have better vision and overall eye health.

In another aspect, the invention provides methods for preventing or ameliorating cloudy eye in animals comprising administering to the animals a cloudy eye preventing or ameliorating amount of one or more polyamines. The polyamines absorb radiation and scavenge free radicals that are at least partially responsible for causing cloudy eye in animals. With no or less severe cloudy eye, the animals have better vision and have a more healthy and appealing appearance. Preferably, the animals are canines and felines, most preferably canines such as dogs.

In a further aspect, the invention provides methods for preventing or ameliorating animal eye injury caused by radiation comprising administering to the animals an eye injury preventing or ameliorating amount of one or more polyamines. The polyamines absorb radiation that damages eye tissue and causes injury to the eye.

In yet another aspect, the invention provides methods for reducing or preventing a decline of social interaction in animals comprising administering to the animals a social interaction decline reducing or preventing amount of one or more polyamines. The polyamines absorb radiation and scavenge free radicals that are at least partially responsible for impairing vision in animals. With better vision, the animals are more socially interactive, particularly when participating in activities that require good vision, e.g., fetch. The methods ensure that the animals remain involved in playtime, participate in group activities, interact with caregivers, and the like.

In one aspect, the invention provides methods for promoting the health and wellness of animals comprising administering to the animals a health and wellness promoting amount of one or more polyamines. The polyamines improve eye health and vision and improve the animal's overall health and wellness.

In another aspect, the invention provides methods for improving the quality of life for animals comprising administering to the animals a quality of life improving amount of one or more polyamines. The polyamines improve eye health and vision and contribute to the quality of life of the animals.

In a further aspect, the invention provides methods for extending the prime for animals comprising administering to the animals a prime extending amount of one or more polyamines. The methods extend the time the animals have good vision and delay the onset of ophthalmic maladies so that the animals have a longer period during their life wherein they have good vision and related ophthalmic functions.

In the methods for maintaining eye health, maintaining or improving vision, preventing or ameliorating cataracts, preventing or ameliorating cloudy eye, preventing or ameliorating animal eye injury caused by radiation, reducing or preventing a decline of social interaction, promoting overall health and wellness, improving the quality of life, and extending the prime, the amount of polyamines used in these methods are the same as the amounts or within the ranges given herein for ameliorating ophthalmic maladies. Administration routes, specific compounds, and other parameters are the same as described herein for ameliorating ophthalmic maladies.

In a further aspect, the invention provides kits suitable for administering polyamines to animals to ameliorate ophthalmic maladies. The kits comprise in separate containers in a single package or in separate containers in a virtual package, as appropriate for the kit component, instructions for how to use polyamines to ameliorate ophthalmic maladies and at least one of (1) one or more polyamines; (2) one or more comestible ingredients containing polyamines; (3) one or more comestible ingredients compatible with polyamines; (4) one or more ophthalmic agents; (5) instructions for how to combine or prepare the polyamines and any other ingredients provided in the kit for administration to animals; and (6) a device for administering the combined or prepared kit components to animals. The components are each provided in separate containers in a single package or in mixtures of various components in different packages. In preferred embodiments, the kits comprise the instructions and one or more polyamines or comestible ingredients containing polyamines. The kits may comprise the ingredients in various combinations.

In another aspect, the invention provides a means for communicating information about or instructions for one or more of (1) using polyamines for ameliorating ophthalmic maladies; (2) using polyamines in conjunction with ophthalmic agents; (3) admixing polyamines with other materials to produce a composition suitable for ameliorating ophthalmic maladies; (4) using the kits of the invention for ameliorating ophthalmic maladies; (5) benefits of using polyamines for ameliorating ophthalmic maladies; and (6) administering polyamines to animals. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the present invention provides packages comprising one or more polyamines or comestible ingredients comprising polyamines or polyamine precursors and a label affixed to the packages containing a word or words, picture, design, acronym, slogan, phrase, or other device, or combination thereof, that indicates that the contents of the package contains polyamines and/or polyamine precursors that are suitable for one or more of ameliorating ophthalmic maladies; maintaining eye health; maintaining or improving vision; preventing or ameliorating cataracts; preventing or ameliorating cloudy eye; preventing or ameliorating animal eye injury caused by radiation; reducing or preventing a decline of social interaction; promoting overall health and wellness; improving the quality of life; and extending the prime for animals. Typically, such device comprises the words ‘prevents damage caused by ultraviolet light, “maintains eye health, “prevents eye damage caused by radiation”, “helps prevent cataracts”, “helps prevent cloudy eye in dogs”, or an equivalent expression printed on the package. Any package or packaging material suitable for containing the polyamines is useful in the invention, e.g., a bag, box, bottle, can, pouch, and the like manufactured from paper, plastic, foil, metal, and the like. In a preferred embodiment, the package contains a food composition adapted for an animal that supplies the required amount of polyamines to the animal, e.g., a pet food composition formulated for animals.

In another aspect, the invention provides for use of one or more polyamines to prepare a medicament for one or more of ameliorating ophthalmic maladies; maintaining eye health; maintaining or improving vision; preventing or ameliorating cataracts; preventing or ameliorating cloudy eye; preventing or ameliorating animal eye injury caused by radiation; reducing or preventing a decline of social interaction; promoting overall health and wellness; improving the quality of life; and extending the prime for animals. Generally, medicaments are prepared by admixing polyamines with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to animals.

### EXAMPLES

The invention can be further illustrated by the following examples, although it will be understood that the examples are included merely for purposes of illustration and are not intended to limit the scope of the invention unless otherwise specifically indicated.

### Example 1

Eye samples were collected from eight (8) senior (10+years old) and eight (8) young (2-3 years old) dogs. Eye balls were removed within 1 hour of death at necropsy, stored in individual containers at 4° C., and dissected within 24 hours. The eyes were dissected to collect the lenses. The lenses were separated into two layers: (1) an outer, soft epithelial/cortical layer and (2) an inner, harder nuclear layer. The lens fractions were analyzed for metabolites. three polyamines, spermidine, spermine, and putrescine were elevated in the young dog lens fractions relative to the senior dog lens fractions. The results are given in Table 1.

TABLE 1
Fold Change In Polyamines In Young Vs. Old Dog Lens Fractions

| | Epithelial/Cortical: Young | Nucleus: Young Vs. |
|---|---|---|
| | Vs. Old Canine | Old Canine |
| Putrescine | 1.45 | 1 |
| Spermidine | 1.79 | 1.71 |
| Spermine | 2.78 | 1 |

Referring to Table 1, the data show that the amount of the polyamines spermine, spermidine, and putrescine are higher in the young dog lenses compared to the senior dog lenses.

### Example 2

Rabbit eyes were obtained from a rabbit renderer and were dissected within twenty-four (24) hours of the animal's death. The lenses were removed aseptically and cultured in M199 media, 20 mM HEPES, 100 units/ml penicillin, 0.1 gm/ml streptomycin and 0.1% BSA under 5% CO₂ at 37° C. in a humidified incubator for twenty-four hours before the start of treatment. Lens opacification was induced with 20 mM CaCl₂, according to the method of Hytower and Dering (Hightower Kr, Dering, M (1984) Invest Ophthalmol Vis Sci 251:108-111). Polyamines were added at a concentration of 100 mM. All experiments were done in triplicate.

Transparency Evaluation: When proteins are modified or oxidized they aggregate, and cause protein solutions to become cloudy or translucent because the proteins lose solubility. The turbidity of a protein solution is a measure of protein aggregation. Turbidity of the lenses after treatment was determined by measuring the increase in protein absorbency. Control (no Ca++), Ca++ and Ca++ with polyamines spermidine, spermine, or putrescine were collected and homogenized in lysis buffer [50 mM Tris-HCl, pH 8.0; 150 mM NaCl, 1% Igepal and protease inhibitor cocktail (Sigma)] after seventy-two (72) hours of treatment. Absorbance was measured at 394 nm for each sample using a spectrophotometer. Transparency was calculated as a function of the percent transmittance according to Jain (Jain Ak, Lim G, Langford M, Jain Sk (2002) Free Radic Biol Med 33:1615-1621). The results are shown in Table 2.

TABLE 2
Percent Light Transmittance After Treatments

| | | | Ca & | Ca & | Ca & |
|---|---|---|---|---|---|
| | Control | Ca | Spermidine | Putrescine | Spermine |
| Average | 73.3 | 47.3 | 56.8 | 65.7 | 53 |
| % Cv | 0.96 | 4.28 | 9.62 | 11.47 | 6.68 |

Referring to Table 2, the data show that the polyamines putrescine and spermidine reduce calcium-induced lens opacification.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

What is claimed is:

1. A method for ameliorating ophthalmic maladies in animals comprising administering to the animals an ophthalmic malady ameliorating amount of one or more polyamines.

2. The method of claim 1 wherein the maladies are caused by aging.

3. The method of claim 2 wherein the maladies are one or more of cloudy eye and cataracts.

4. The method of claim 1 wherein the maladies are caused by radiation.

5. The method of claim 4 wherein the maladies are one or more of cataracts, radiation retinopathy, photokeratitis, photoconjunctivitis, pterygium, pinguecula, and cancer.

6. The method of claim 1 wherein the maladies are caused by free radicals.

7. The method of claim 6 wherein the maladies are one or more of cloudy eye and cataracts.

8. The method of claim 1 wherein the polyamines are cadaverine, spermidine, spermine, putrescine, and combinations thereof.

9. The method of claim 1 wherein the polyamines are spermidine, spermine, putrescine, and combinations thereof.

10. The method of claim 1 wherein the polyamines are spermidine, spermine, and combinations thereof.

11. The method of claim 1 wherein the polyamines are administered to the animals by administering a polyamine precursor to the animals and allowing the polyamines to be synthesized in vivo.

12. The method of claim 11 wherein the polyamine precursor is arginine, citrulline, ornithine, or combinations thereof.

13. The method of claim 1 wherein the polyamines are administered to the animals in amounts of from about 1 to about 200 mg/kg/day.

14. The method of claim 1 wherein the polyamines are administered to the animals in amounts of from about 10 to about 180 mg/kg/day.

15. The method of claim 1 wherein the animals are canines.

16. The method of claim 1 further comprising administering the polyamines in conjunction with one or more ophthalmic agents in an amount effective for ameliorating ophthalmic maladies.

17. A method for preventing or ameliorating cataracts in animals comprising administering to the animals a cataract preventing or ameliorating amount of one or more polyamines.

18. A method for preventing or ameliorating cloudy eye in animals comprising administering to the animals a cloudy eye preventing or ameliorating amount of one or more polyamines.

19. A method for preventing or ameliorating animal eye injury caused by radiation comprising administering to the animals an eye injury preventing or ameliorating amount of one or more polyamines.

20. A use of polyamines to prepare a medicament for one or more of ameliorating ophthalmic maladies; maintaining or improving vision; preventing or ameliorating cataracts; preventing or ameliorating cloudy eye; preventing or ameliorating animal eye injury caused by radiation; reducing or preventing a decline of social interaction; promoting overall health and wellness; improving the quality of life; and extending the prime for animals.
